(19) United States
(12) Patent Application Publication
(10) Pub. No.: US 2010/0224072 A1
(43) Pub. Date: Sep. 9, 2010

(54) System for purification of air in an inner space

(76) Inventor: Gennady N. Ulanov, Brooklyn, NY (US)

Correspondence Address: Ilya Zborovsky, 6 Schoolhouse Way, Dix Hills, NY, 11746, US

(21) Appl. No.: 12/380,659

(22) Filed: Mar. 3, 2009

(51) Int. Cl. F24F 13/00; B01D 46/42; B01D 46/00; F24F 6/00; A61L 9/00

(52) U.S. Cl. 96/222; 55/410; 55/410.1; 96/234; 96/235; 96/240; 96/424

(57) ABSTRACT

A system for purification of air in an inner space has a housing having air inlet unit and air outlet units; air filtering means located in the housing between the air inlet unit and the air outlet units; and vertical attachment attached to the air outlet units and extending vertically upwardly so that air enters the housing at a lower level through the air inlet units and leaves the system at an outlet of the attachment at a level substantially higher than the level of the inlet units.

### BACKGROUND OF THE INVENTION

The present invention relates to a system for purification of air in an inner space.

There are many systems of the above mentioned general type. However, it is believed that they can be further improved in the sense of increasing the purification quantity of air in providing additional purification aspects.

### SUMMARY OF THE INVENTION

Accordingly, it is an object of the present invention to provide a system for purification of air in a space, which is a further improvement of the existing systems.

In keeping with these objects and with others which will become apparent, one feature of the present invention resides, briefly stated, in a system for purification of air in an inner space, comprising a housing having air inlet means and air outlet means; air filtering means located in said housing between said air inlet means and said air outlet means; and vertical attachment attached to said air outlet means and extending vertically upwardly so that air enters said housing at a lower level through said air inlet means and leaves said system at an outlet of said attachment at a level substantially higher than the level of said inlet means.

The novel features which are considered as characteristic for the present invention are set forth in particular in the appended claims. The invention itself, however, both as to its construction and its method of operation, together with additional objects and advantages thereof, will be best understood from the following description of specific embodiments when read in connection with the accompanying drawings.

### BRIEF DESCRIPTION OF THE DRAWINGS

FIG. 1 is a plan view of a system for purification of air in an inner space;

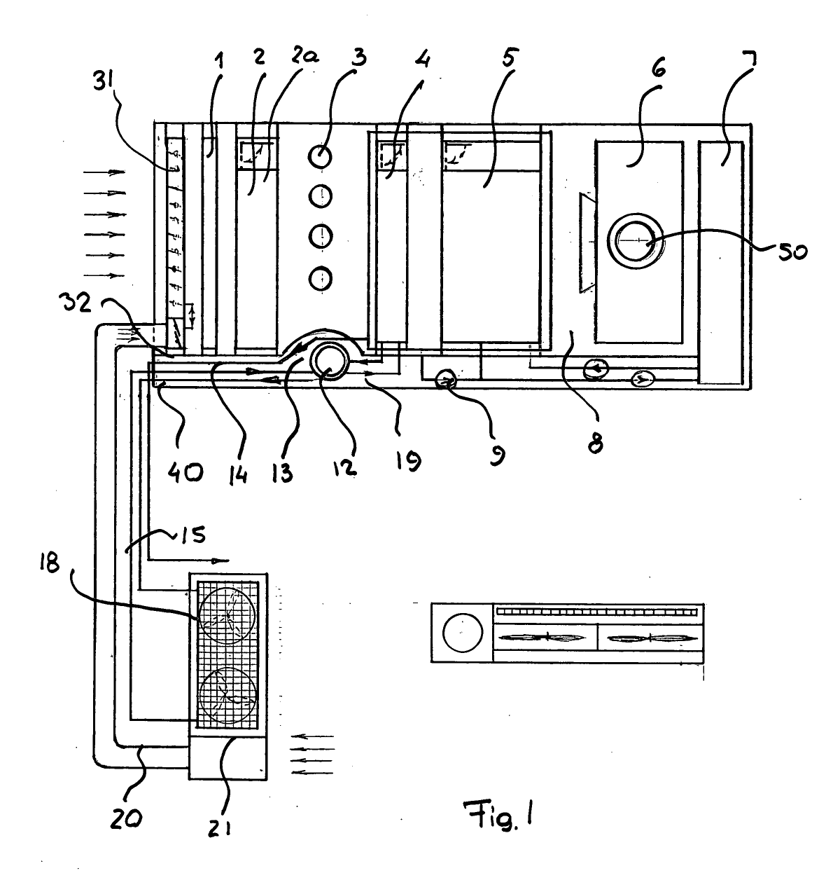

FIG. 2 is a side view of the inventive system for purification of air in an inner space;

FIG. 3 is a view showing an air humidifier of the system in accordance with the present invention; and

FIG. 4 is a perspective view of the inventive system.

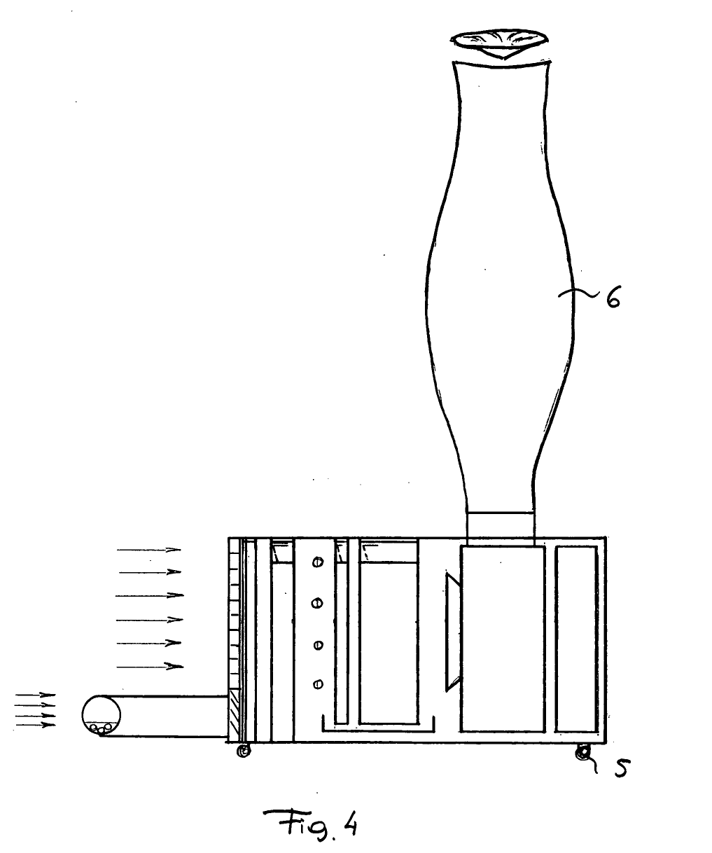

### DESCRIPTION OF THE PREFERRED EMBODIMENTS

A system for purification of air in an inner space in accordance with the present invention has a housing identified with reference 30 and provided with an inlet 40 and an outlet 50 for entry of air into the housing and exit of air from the housing.

Filtering means are located in the housing and can include a prefilter 1 and a HEPA filter 2. Flaps can be provided on a periphery of the HEPA filter 2, so that when they are open air goes through prefilter 1 and around the HEPA filter, and when the flaps close the passage between the HEPA filter and the housing, the air also flows through the HEPA filter.

An ultraviolet device 3 is located after the filtering means and emits ultraviolet radiation to purify the air by killing germs, etc.

After the ultraviolet device an air conditioner 4 is located which cools the air passing from the inlet to the outlet through the housing.

A fan 6 provides suction of air from the inlet to the outlet through the filtering means 1, 2, the ultraviolet device 3, air conditioner 4, and a humidifier 5.

The humidifier 5 is connected with a water container 7 through a pipe. Another pipe connects the outlet of the air conditioner 4 with the water container 7 to supply a discharge water from the air conditioner 4 into the water container. A filter can be located in this pipe, and an additional pump can move water from the air conditioner 4 into the water container 7. Reference numeral 9 identifies a tray for collecting water (condensed water) from the air conditioner. The pipe for supplying the condensed water into water container is identified with reference numeral 7 and the corresponding pump is identified with reference numeral 11.

The air conditioner 4 has a compressor which pumps a cooling fluid through the air conditioner which is heated by the passing air, and for sucking the heated cooling fluid, for example Freon.

The system further has a heat exchanger which is identified with reference numeral 13. It provides a heat exchange between the heated cooling fluid from the air conditioner and a cool cooling liquid, which cools the heated cooling fluid.

Reference numeral 14 identifies a valve for ventilation, to supply a fresh air from outside.

The system further has a flexible pipe or hose from taking air, in which the allocated a pipe 17 for supplying a cold water to the heat exchanger and withdrawing a heated water from the heat exchanger. The cooling water is cooled by a cooler or radiator 20 provided with two ventilators 21 which can operate individually or jointly in accordance with a program.

Reference numeral 18 identifies a pipe for withdrawing an excess of water and reference numeral 19 identifies a pump for displacing the excessive water.

An additional exterior air can be supplied through the pipe 15 into the interior of the housing.

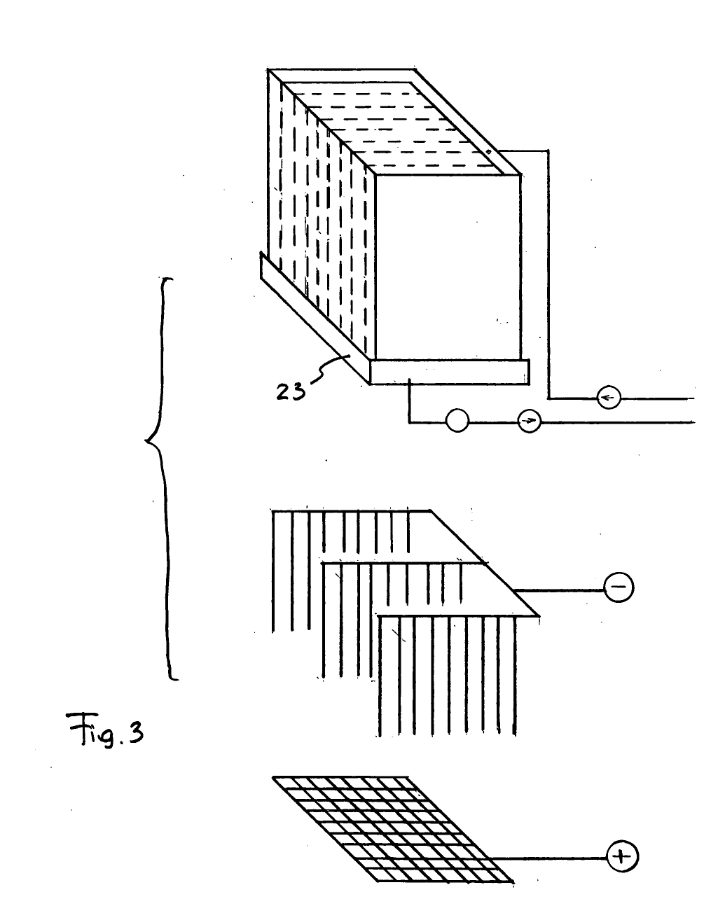

The air humidifier 5 shown in FIG. 3 has a negative electrode 22 formed by a plurality of silver wires located on sections of the humidifier, and a positive electrode 23 which is formed by a bottom net. A circulating pump 24 is further provided for supplying water. There is a gap between the silver wires of the negative electrode and the net of the positive electrode. This gap is breached by water when it flows through the humidifier, thus causing movement of negative ions of silver from the negative electrode to the positive electrode and impregnating water with silver ions, that provide bactericidal properties to water.

A control unit 25 controls the operation of the inventive system. It can turn on and off of each individual unit of the system by an operator, or remotely, and even through an internet.

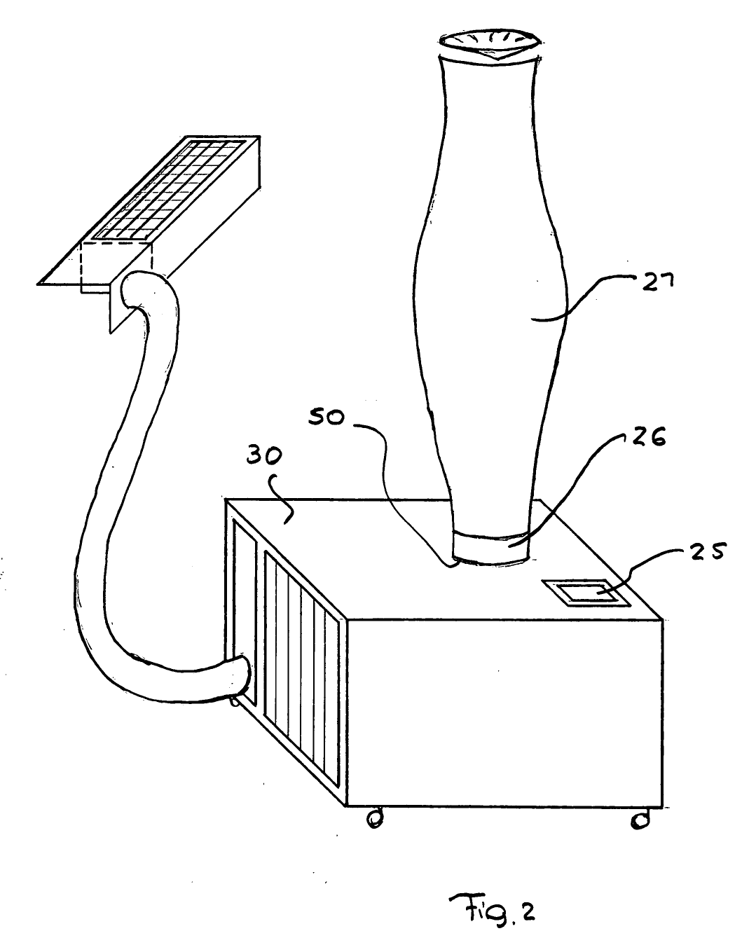

As shown in FIG. 2, the system further has an aroma producing element which is identified with reference numeral 26 and located in the area of the outlet means 50 of the system. The outlet means 50 is connected with an attachment 27 which is formed as a vertical attachment. The vertical attachment 27 can have a height substantially between 3′ and 10′. With the vertical attachment the air enters the housing at a lower level, and leaves the housing at a substantially higher level through an outlet of the attachment 27, thus providing a significant advantage in proper purification of air.

The attachment 27 can be formed as a lamp, a sculpture, a column, a tree, fallows, etc.

In the system either air from a room or outside air can be introduced into the housing. When flaps 31 are open and a valve 32 is closed, air from the room is introduced into the housing, while when the flaps 31 are closed and the valve 32 is open air from outside is introduced into the housing into the housing through the pipe 15.

It will be understood that each of the elements described above, or two or more together, may also find a useful application in other types of constructions differing from the type described above.

While the invention has been illustrated and described as embodied in a system for purification of air in an inner space, it is not intended to be limited to the details shown, since various modifications and structural changes may be made without departing in any way from the spirit of the present invention.

Without further analysis, the foregoing will so fully reveal the gist of the present invention that others can, be applying current knowledge, readily adapt it for various applications without omitting features that, from the standpoint of prior art, fairly constitute essential characteristics of the generic or specific aspects of this invention.

What is claimed as new and desired to be protected by Letters Patent is set forth in the appended claims.

1. A system for purification of air in an inner space, comprising a housing having air inlet means and air outlet means; air filtering means located in said housing between said air inlet means and said air outlet means; and vertical attachment attached to said air outlet means and extending vertically upwardly so that air enters said housing at a lower level through said air inlet means and leaves said system at an outlet of said attachment at a level substantially higher than the level of said inlet means.

2. A system as defined in claim 1, wherein said attachment has a height of substantially between 3 and 10 feet.

3. A system as defined in claim 1, wherein said filtering means include a prefilter and a HEPA filter.

4. A system as defined in claim 1; and further comprising a conditioner which calls the air conditioner and located in said housing between said inlet means and said outlet means.

5. A system as defined in claim 4, wherein said air conditioner is operative with a cooling fluid which cools air and is heated by air; and further comprising a cooling liquid contours through which a cooling liquid flows and which is cooled by an outside air and provides cooling of the heated cooling fluid in said air conditioning.

6. A system as defined in claim 5, wherein said cooling contour includes a heat exchanger providing a heat exchange between the cooling liquid and the heated cooling fluid of said air conditioner.

7. A system as defined in claim 1, wherein said cooling contour includes an outside hose through which an outside air enters said housing.

8. A system as defined in claim 1; and further comprising a cooler of the cooling liquid which is heated by the heated cooling fluid, and at least two ventilators which can operate in the independently of one another and jointly with one another.

9. A system as defined in claim 1; and further comprising a humidifier of air located in said housing between said inlet means and said outlet means and humidifying the air passing from said inlet means to said outlet means.

10. A system as defined in claim 9, wherein said humidifier includes silver elements forming a negative electrode and a net forming a positive electrode which are spaced from one another, but become electrically connected with one another when water passes between said negative electrode and said positive electrode so that silver can migrate into the water.

11. A system as defined in claim 9; and further comprising a water container for supplying said water humidifier.

12. A system as defined in claim 11, wherein said air conditioner is connected with said container so that a spray of water from said air conditioner flows into said container.

13. A system as defined in claim 12; and further comprising a filter located between said air conditioner and said water container for purifying water which flows from said air conditioner to said container.

14. A system as defined in claim 1; and further comprising control means for controlling of the system, so that each individual unit can operate at a certain time and over a certain period of time.

15. A system as defined in claim 1; and further comprising means located in said housing and operated for adding aroma to the air passing from said inlet means to said outlet means.

16. A system as defined in claim 15, wherein said aroma adding means is configured so as to provide different aromas into the air.

17. A system as defined in claim 1; and further comprising means allowing a supply of a room air or a supply of an outside air through said inlet means into said housing.
